(19) United States
(12) Patent Application Publication
(10) Pub. No.: US 2020/0129185 A1
(43) Pub. Date: Apr. 30, 2020

(54) IN-VIVO INDWELLING INSTRUMENT AND IN-VIVO INDWELLING INSTRUMENT DELIVERING SYSTEM

(71) Applicant: KANEKA CORPORATION, Osaka-shi, Osaka (JP)

(72) Inventor: Hidekazu NAKANISHI, Settsu-shi (JP)

(73) Assignee: KANEKA CORPORATION, Osaka-shi, Osaka (JP)

(21) Appl. No.: 16/621,891

(22) PCT Filed: Apr. 27, 2018
(86) PCT No.: PCT/JP2018/017218

§ 371 Date: Dec. 12, 2019

(30) Foreign Application Priority Data
Jul. 31, 2017 (JP) 2017-147759

(51) Int. Cl. A61B 17/12

(52) U.S. Cl. CPC A61B 17/12154; A61B 2017/00853; A61B 2017/1205; A61B 17/12113

(57) ABSTRACT

An in-vivo indwelling instrument in which a stretch resistant member at a distal end part of a coil does not easily stray from the coil, and a delivering system for the in-vivo indwelling instrument is disclosed. An in-vivo indwelling instrument includes a coil formed by winding a wire, and a stretch resistant member that is placed in a lumen of the coil. In the view of the coil from a distal side, a part of the wire exists within a central region surrounded by a circle having a diameter of half a maximum outer diameter of the coil and having a center that is a middle point of the maximum outer diameter. The stretch resistant member and a connecting part which is the part of the wire are connected to each other.

### TECHNICAL FIELD

The present invention relates to an in-vivo indwelling instrument for vascular embolization at a vascular disease site in a blood vessel and an in-vivo indwelling instrument delivering system.

### BACKGROUND ART

In an endovascular treatment, which is one of treatment methods of vascular lesions such as an aneurysm of a head and neck, an arteriovenous malformation, an arteriovenous fistula, a pulmonary vascular malformation, a renovascular malformation, an aneurysm of renal artery, and an abdominal aneurysm, an in-vivo indwelling instrument for embolization is indwelled at a target site to promote the thrombogenesis, thereby preventing a rupture of the aneurysm, for example.

Patent Literatures 1 to 4 disclose an in-vivo indwelling instrument for embolization including a coil, a stretch resistant member placed in the coil, and a tip member placed at a distal end part of the coil. The in-vivo indwelling instrument is attached to a tip end part of a pusher, and pushed by the pusher toward a distal side of a catheter or the like that is used when the instrument is indwelled, whereby the in-vivo indwelling instrument is delivered to a target site in the body, such as the aneurysm.

### CITATION LIST

### Patent Literature

Patent Literature 1 Japanese Translation of PCT International Application Publication No. JP-T-2008-525113
Patent Literature 2 Japanese Unexamined Laid-open Patent Application Publication No. 2016-154946
Patent Literature 3 Japanese Unexamined Laid-open Patent Application Publication No. 2012-464
Patent Literature 4 WO 2010/123003

### SUMMARY OF THE INVENTION

### Problems to be Solved by the Invention

In the in-vivo indwelling instrument disclosed in Patent Literatures 1 to 4, if the strength of a distal end part is low, the stretch resistant member may possibly stray from the coil when the in-vivo indwelling instrument is pushed by a pusher, which may make it impossible for the indwelling instrument to be delivered to a target site. In view of the above, it is an object of the present invention to provide an in-vivo indwelling instrument in which a stretch resistant member at a distal end part of a coil does not easily stray from the coil, and a delivering system for the in-vivo indwelling instrument.

### Solution to the Problems

An in-vivo indwelling instrument which is able to achieve the above object includes: a coil that is formed by winding a wire, and extends in a distal and proximal direction; and a stretch resistant member that is placed in a lumen of the coil. In the view of the coil from a distal side, a part of the wire exists within a central region surrounded by a circle having a diameter of half a maximum outer diameter of the coil and having a center that is a middle point of the maximum outer diameter. The stretch resistant member and a connecting part which is the part of the wire are connected to each other. According to the in-vivo indwelling instrument of the present invention, a part of a wire (connecting part) is connected to a stretch resistant member. Therefore, it is possible to inhibit the stretch resistant member from straying from the coil. Further, the part of the wire (connecting part) is provided within a central region, and therefore, the stretch resistant member connected to the connecting part is easily disposed within the central region. Thus, a function of inhibiting the stretch resistant member from extending in an axial direction of the coil can be effectively exerted.

In the in-vivo indwelling instrument, a proximal end of the connecting part is preferably disposed more distally than a position having a length of one-tenth of an entire length of the coil from the distal end of the coil toward a proximal side.

In the in-vivo indwelling instrument, the wire preferably includes a part having a curvature radius smaller than a curvature radius of the coil and the part being placed between the coil and the connecting part.

In the in-vivo indwelling instrument, in the view of the coil from the distal side, the wire includes a part that has a form of a closed curve, and an area surrounded by the closed curve is preferably 75% or less of an area surrounded by a circumference of the coil.

In the in-vivo indwelling instrument, it is preferable that the connecting part is a part in which the wire is folded back in a shape of a hook along the distal and proximal direction of the coil.

In the in-vivo indwelling instrument, a tip end of the wire preferably exists outside the central region.

In the in-vivo indwelling instrument, a tip end of the wire preferably exists inside the central region.

In the in-vivo indwelling instrument, in the view of the coil from the distal side, at the connecting part a part of the wire preferably extends through the center of the central region.

In the in-vivo indwelling instrument, the stretch resistant member preferably has a wave shape.

In the in-vivo indwelling instrument, an amplitude of a wave of the stretch resistant member is preferably equal to or larger than an outer diameter of the wire.

In the in-vivo indwelling instrument, a tip member is preferably connected to a distal end part of the coil.

In the in-vivo indwelling instrument, the tip member is preferably made of an ultraviolet curing resin.

The present invention provides an in-vivo indwelling instrument delivering system including: the in-vivo indwelling instrument; a detachment part connected to a proximal end part of the in-vivo indwelling instrument; and a pusher part connected to the coil of the in-vivo indwelling instrument through the detachment part.

### Effects of the Invention

According to an in-vivo indwelling instrument of the present invention, it is possible to inhibit the stretch resistant member from straying from the coil. Further, the stretch resistant member is easily disposed within the central region, and therefore, a function of inhibiting the stretch resistant member from extending in an axial direction of the coil can be effectively exerted.

### BRIEF DESCRIPTION OF THE DRAWINGS

FIG. 1 is a cross-sectional view (a partial side view) of an in-vivo indwelling instrument according to an embodiment of the present invention.

FIG. 2 is a front view of a coil of the in-vivo indwelling instrument illustrated in FIG. 1.

FIG. 3 is a front view showing a modification example of the coil of the in-vivo indwelling instrument illustrated in FIG. 2.

FIG. 4 is a cross-sectional view (a partial side view) of the coil according to an embodiment of the present invention.

FIG. 5 is a front view of the coil illustrated in FIG. 4.

FIG. 6 is a side view of an in-vivo indwelling instrument delivering system according to an embodiment of the present invention.

### MODE FOR CARRYING OUT THE INVENTION

Hereinafter, the present invention will be specifically explained below based on the following embodiments; however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

An in-vivo indwelling instrument according to the present invention includes: a coil that is formed by winding a wire, and extends in a distal and proximal direction; and a stretch resistant member that is placed in a lumen of the coil. In the view of the coil from a distal side, a part of the wire exists within a central region surrounded by a circle having a diameter of half a maximum outer diameter of the coil and having a center that is a middle point of the maximum outer diameter. The stretch resistant member and a connecting part which is the part of the wire are connected to each other.

FIG. 1 is a cross-sectional view (a partial side view) of an in-vivo indwelling instrument 10 according to an embodiment of the present invention. FIG. 2 is a front view of a coil 11 of the in-vivo indwelling instrument 10 illustrated in FIG. 1. FIG. 1 illustrates a state in which the in-vivo indwelling instrument 10 expands linearly. The in-vivo indwelling instrument 10 includes a distal side and a proximal side. The proximal side of the in-vivo indwelling instrument 10 indicates a side in a direction toward a user's (operator's) hand with respect to an extending direction of the in-vivo indwelling instrument 10, and the distal side indicates a side in a direction opposite to the proximal side (i.e., in a direction toward a procedure target). Furthermore, a direction from the proximal side to the distal side of the in-vivo indwelling instrument 10 is referred to as an axial direction or a distal and proximal direction. The in-vivo indwelling instrument 10 includes a coil 11 that is formed by winding a wire 12 and extends in the distal and proximal direction, and a stretch resistant member 21 that is placed in a lumen of the coil 11. The part at which the wire 12 is wound into a coil shape is referred to as the coil 11.

The coil 11 is formed by one or a plurality of wires 12 being wound spirally, and is, for example, a secondary coil formed by a primary coil, which is formed by the wires 12 being wound spirally, being further wound, for example, spirally or in a three-dimensional shape, around the primary coil. In FIG. 1, the coil 11 is illustrated in a manner that the secondary coil is extended linearly for the purpose of easy understanding of the shape of the primary coil. The density (winding interval) of the coil 11 is not specifically limited, and the coil 11 can be formed by tight winding, pitch winding, or a combination thereof. In the coil 11, parts of the wire 12 that are adjacent to each other may be in contact with each other at least at a part of the in-vivo indwelling instrument 10 in the distal and proximal direction, or alternatively, parts of the wire 12 that are adjacent to each other may be in contact with each other entirely in the distal and proximal direction. In the wound wire 12 of the primary coil, the state in which the parts of the wire 12 that are adjacent to each other in the distal and proximal direction are in contact with each other is referred to as the tight winding, and the state in which the parts of the wire 12 that are adjacent to each other in the distal and proximal direction are not in contact with each other is referred to as the pitch winding. Such a state of not being in contact with each other refers to a state in which the parts of the wire 12 that are adjacent to each other in the distal and proximal direction are separated from each other.

The wire 12 forming the coil 11 preferably has bio-compatibility and flexibility, and is, for example, more preferably made of metal materials such as platinum, gold, titanium, tungsten, and alloys thereof, and stainless steel, and is yet more preferably made of, namely, a platinum-tungsten alloy.

The cross-sectional shape of the wire 12 forming the coil 11 in the axial direction may be circle, ellipse, polygon, or a combination thereof. The coil 11 may be a single-layer coil, or alternatively, may be a multi-layer coil having a plurality of layers. For example, a chemical may be applied to at least either one of the coil 11, the wire 12, and the stretch resistant member 21.

The outer diameter of the wire 12 forming the coil 11 is not particularly limited, and, for example, may be 25 μm or more, 30 μm or more, or 35 μm or more, and even may be 75 μm or less or 70 μm or less, as an acceptable outer diameter. The wire 12 may be a single wire-shaped member from one end to the other end, or alternatively, may be provided by a plurality of wire-shaped members being coupled to each other.

The stretch resistant member 21 is a wire-shaped member that inhibits the coil 11 from extending in the axial direction during the operation of the in-vivo indwelling instrument 10. The stretch resistant member 21 may be a single wire or a stranded wire. Furthermore, the stretch resistant member 21 may has a single layer or may be a multi-layer body having a plurality of layers. For example, the stretch resistant member 21 may include an inner layer made of a stranded wire having a plurality of wires 12 and an outer layer located on the outer side of the inner layer and including resin composition. A single piece or a plurality of pieces of the stretch resistant member 21 may be placed in the coil 11.

The stretch resistant member 21 is preferably made of resin or a metal material. For example, examples of the metal material include platinum, gold, rhodium, palladium, rhenium, gold, silver, titanium, tantalum, tungsten and alloys thereof, and stainless steel, and examples of the resin material include polyester resin such as polyethylene terephthalate, polyamide resin such as nylon, polyolefin resin such as polyethylene, polypropylene, and the like. As long as the stretch resistant member 21 is made of resin, the stretch resistant member 21 can increase flexibility and improve the delivery performance of the in-vivo indwelling instrument 10. Furthermore, the stretch resistant member 21 made of resin is not ruptured due to metal fatigue during delivery, and can mitigate stretching by the end part of the coil 11 extending linearly due to the insufficient length of the stretch resistant member 21 when the coil 11 is placed in an aneurysm. The stretch resistant member 21 may be made of a material different from the coil 11. For example, the coil 11 is preferably made of a platinum-tungsten alloy, and the stretch resistant member 21 is preferably made of polypropylene resin.

The cross-sectional shape of the stretch resistant member 21 in the axial direction may be circle, ellipse, polygon, or a combination thereof. The outer diameter of the stretch resistant member 21 suffices as long as it is smaller than a lumen of the coil 11. As described later, the stretch resistant member 21 is preferably placed in the lumen of the coil 11 in a state being folded back. Therefore, the outer diameter of the stretch resistant member 21 is preferably smaller than one second an inner diameter of the coil 11, and more preferably one third or less. In order to prevent a rupture of the stretch resistant member 21, the outer diameter of the stretch resistant member 21 is preferably one fifteenth or more the inner diameter of the coil 11, and more preferably, one tenth or more. The outer diameter of the stretch resistant member 21 may be, for example, 20 μm or more, 25 μm or more, or 40 μm or less, or 35 μm or less.

As illustrated in FIG. 1, the stretch resistant member 21 is connected to the connecting part 25 of the coil 11. The coil 11 and the connecting part 25 are formed by the same wire 12. The coil 11 is a part at which the wire 12 is wound in a coil shape, and the connecting part 25 is a part of the wire 12. In a case in which the coil 11 is caused to extend in the distal and proximal direction, the connecting part 25 is disposed more distally than the middle point in the distal and proximal direction. The stretch resistant member 21 and the connecting part 25 of the coil 11 can be connected and fixed to each other by, for example, a physically fixing method such as deposition, welding, clamping such as caulking, adhesion by an adhesive, engaging, coupling, binding, knotting, or a combination thereof.

As illustrated in FIG. 1, the stretch resistant member 21 includes a folded part 21a that is folded back in the distal and proximal direction, and the folded part 21a is preferably connected to the connecting part 25 of the wire 12. Specifically, the folded part 21a of the stretch resistant member 21 is preferably hooked at the connecting part 25 of the wire 12. With such a configuration, the stretch resistant member 21 and the connecting part 25 can be readily connected to each other. This makes it possible to simplify the producing process of the in-vivo indwelling instrument 10.

The stretch resistant member 21 is preferably fixed to a proximal end part of the coil 11 as well. The stretch resistant member 21 and the proximal end part of the coil 11 can be fixed to each other by the similar way to the connection between the stretch resistant member 21 and the connecting part 25. The stretch resistant member 21 can also be fixed to a connecting part of the coil 11 and a pusher or a detachment part 2, in addition to the proximal end part of the coil 11. In FIG. 1, the proximal end part of the stretch resistant member 21 and the distal end part of the detachment part 2 are bound together.

The stretch resistant member 21 preferably has a linear shape, a wave shape, or a spiral shape, and more preferably has the wave shape. This makes it possible to smoothly indwell the stretch resistant member 21 until the end of the coil 11 and secure the length of the stretch resistant member 21 in the interior of the coil 11. Therefore, it is possible to mitigate a phenomenon of stretching by the end part of the coil 11 extending linearly due to the insufficient length of the stretch resistant member 21 can be mitigated when the in-vivo indwelling instrument 10 is indwelled at a target site during treatment. In a case in which the stretch resistant member 21 has a wave shape, the amplitude of the wave of the stretch resistant member 21 is preferably equal to or larger than the outer diameter of the wire 12. Setting the amplitude in this way allows the stretch resistant member 21 to be readily hooked at the wire 12, as compared with the case in which the stretch resistant member 21 is in a linear shape. This allows for the easy connection of the stretch resistant member 21 and the connecting part 25 of the coil 11 during producing. Furthermore, since the stretch resistant member 21 substantially becomes longer, it is possible to further mitigate the insufficiency of the length of the stretch resistant member 21. The amplitude of the wave of the stretch resistant member 21 may be 25 μm or more, 30 μm or more, or 40 μm or more, or may be 100 μm or less, 80 μm or less, or 60 μm or less.

Moreover, a tip member 20 is preferably connected to the distal end part of the coil 11. The tip member 20 is a member that covers the distal end part of the coil 11 to prevent the tip end 12a of the wire 12 from being in direct contact with the inner wall of a blood vessel. With the conventional in-vivo indwelling instruments, the tip member 20 is essentially provided for the purpose of fixing the stretch resistant member 21 at the tip end of the in-vivo indwelling instrument 10. On the contrary, in the present invention, since the connecting part 25 of the coil 11 and the stretch resistant member 21 are preferably connected directly to each other, a mode in which the tip member 20 is not connected to the stretch resistant member 21 is also accepted. The shape of the tip member 20 is not particularly limited, but can be formed in, for example, a hemispherical shape, a semi-ellipse spherical shape, columnar shape, or polygonal pillar shape.

The tip member 20 is preferably made of a metal material, or a resin such as a thermoplastic resin and an ultraviolet curing resin, and, among them, more preferably made of ultraviolet curing resin without requiring any heat source. As a resin, epoxy acrylate resin, urethane acrylate resin, or polyester acrylate resin can be used. The viscosity of a resin constituting the tip member 20 may be 10 mPa·s or more, 50 mPa·s or more, or 100 mPa·s or more, and even may be 2000 mPa·s or less, 1500 mPa·s or less, or 1000 mPa·s or less, as an acceptable viscosity. Furthermore, the melt flow rate of a resin constituting the tip member 20 may be 0.1 g/min or more, 1 g/min or more, 10 g/min or more, or 25 g/min or more, and may also be 100 g/min or less, 75 g/min or less, or 50 g/min or less, as a melt flow rate.

In order to inhibit the tip member 20 from being unintentionally drawn into the proximal side, the outer diameter of the tip member 20 is preferably larger than the inner diameter of the coil 11. Furthermore, in order to prevent the tip member 20 from straying from the coil 11, a part of the tip member 20 is preferably placed in the lumen of the coil 11, and is more preferably inserted into the lumen of the distal end part of the coil 11.

The proximal end of the tip member 20 is preferably disposed more distally than the position having a length of one tenth of the entire length of the coil 11 from the distal end of the coil 11 toward the proximal side, more preferably disposed more distally than the position having a length of one fifteenth, and yet more preferably disposed more distally than the position having a length of one twentieth. By setting the position of the tip member 20 in this way, the flexible in-vivo indwelling instrument 10 that is suited for the finishing process by a procedure of an endovascular treatment is obtained.

As illustrated in FIG. 1, the tip member 20 is preferably joined with an inner surface of the coil 11. In such a case, the tip member 20 preferably exists closer to the proximal side than the tip end 12a of the wire 12. Since the tip member 20 is fixed firmly to the coil 11, it is possible to inhibit the tip member 20 from straying from the coil 11.

The tip member 20 preferably extends more distally than the distal end of the coil 11. A part extending more distally than the distal end of the coil 11 is assumed as a tip end part 20a of the tip member 20. The tip end part 20a of the tip member 20 preferably has a size that is two times or more the outer diameter of the wire 12 in the axial direction, more preferably three times or more, yet more preferably four times or more, or has a size that is seven times or less or six times or less, as an acceptable size. Setting the length of the tip end part 20a of the tip member 20 in this way makes it possible to cover the distal end of the coil 11 to avoid damage on an inner wall of a blood vessel while securing the flexibility of the distal end part of the in-vivo indwelling instrument 10.

As illustrated in FIG. 2, in the view of the coil 11 from a distal side, a part of the wire 12 exists within a central region 15 surrounded by a circle having a diameter of half the maximum outer diameter of the coil 11 and having a center that is the middle point 11a of the maximum outer diameter. Furthermore, the stretch resistant member 21 is connected to the connecting part 25 which is the part of the wire 12 existing within the central region 15. Since the connecting part 25 of the wire 12 is connected to the stretch resistant member 21 in this way, it is possible to inhibit the stretch resistant member 21 from straying from the coil 11 when the in-vivo indwelling instrument 10 is pushed by a pusher part 3. Furthermore, since the connecting part 25 of the wire 12 exists within the central region 15, the stretch resistant member 21 that is connected to the connecting part 25 is also readily disposed in the central region 15. This allows the function of inhibiting the stretch resistant member 21 from extending in the axial direction of the coil 11 to be effectively exerted.

As illustrated in FIGS. 1 and 2, the connecting part 25 of the wire 12 is preferably connected directly to the stretch resistant member 21. This further inhibits the stretch resistant member 21 from straying from the coil 11. Although not illustrated, the connecting part 25 of the wire 12 may be connected to the stretch resistant member 21 through another member.

The shape of the connecting part 25 of the wire 12 is not particularly limited as long as the connecting part 25 exists within the central region 15. In the view of the coil 11 from the distal side, the connecting part 25 of the wire 12 preferably extends in a direction different from a circumferential direction of the coil 11. For example, as illustrated in FIG. 2, the wire 12 preferably includes a large curvature part 26 (26A) that is a part having a curvature radius smaller than a curvature radius of the coil 11 and the part being placed between the coil 11 and the connecting part 25. In the large curvature part 26A, since a curvature indicating a curved degree of a curved line is large, the curvature radius is small. Providing the large curvature part 26A in this way allows a part of the wire 12 that is disposed more distally than the large curvature part 26A to readily extend in the central region 15. Therefore, by hooking the stretch resistant member 21 at the part of the wire 12 that is disposed more distally than the large curvature part 26A, the connecting part 25 can be disposed at the part of the wire 12 that is disposed more distally than the large curvature part 26A. Such a part to hook the stretch resistant member 21 of the wire 12 is the connecting part 25.

In the view of the coil 11 from the distal side, the wire 12 preferably includes one or a plurality of the large curvature parts 26. In FIG. 2, for the wire 12, two large curvature parts 26A and 26B are provided. A part between the two large curvature parts 26A and 26B preferably extends in the central region 15. Since this makes it possible to provide the connecting part 25 between the two large curvature parts 26A and 26B, the stretch resistant member 21 is readily connected to the central region 15. Such a part between the two large curvature parts 26A and 26B of the wire 12 is the connecting part 25.

FIG. 2 illustrates the example in which the large curvature parts 26 are provided at two positions of the wire 12 in the view of the coil 11 from the distal side. However, three or more of the large curvature parts 26 may be provided so that the wire 12 is formed in a spiral shape. Since a spiral part becomes the connecting part 25, the stretch resistant member 21 is readily connected to the central region 15.

From the viewpoint of easy processing of the coil 11, as illustrated in FIG. 1, the tip end 12a of the wire 12 may be disposed at the distal end of the coil 11. In the view of the coil 11 from the distal side, the tip end 12a of the wire 12 is visually recognized. In the present mode, the tip member 20 is preferably provided at the distal end part of the coil 11 in order to inhibit the tip end 12a of the wire 12 from being in direct contact with a wall of a blood vessel.

The tip end 12a of the wire 12 may be disposed more proximally than the distal end of the coil 11. The tip end 12a of the wire 12 may be in contact with the coil 11 (more preferably, a part of the coil 11 around which the wire 12 is wound). Although not illustrated, the tip end 12a of the wire 12 may be disposed at a separating part at which the parts of the wire 12 of the wound coil 11 that are adjacent to each other are separated from each other. In such a case, the parts of wire 12, 12 disposed at the both sides of the separating part are preferably in contact with the tip end 12a of the wire 12 with each other. Providing the tip end 12a of the wire 12 in this way makes it difficult for the stretch resistant member 21 to fall off from the connecting part 25 of the wire 12.

As illustrated in FIG. 2, in the view of the coil 11 from the distal side, at the connecting part 25 a part of the wire 12 preferably extends through the center of the central region 15. Forming the wire 12 in this way allows the stretch resistant member 21 that is connected to the connecting part 25 of the wire 12 to be readily disposed in the proximity of the center of the central region 15.

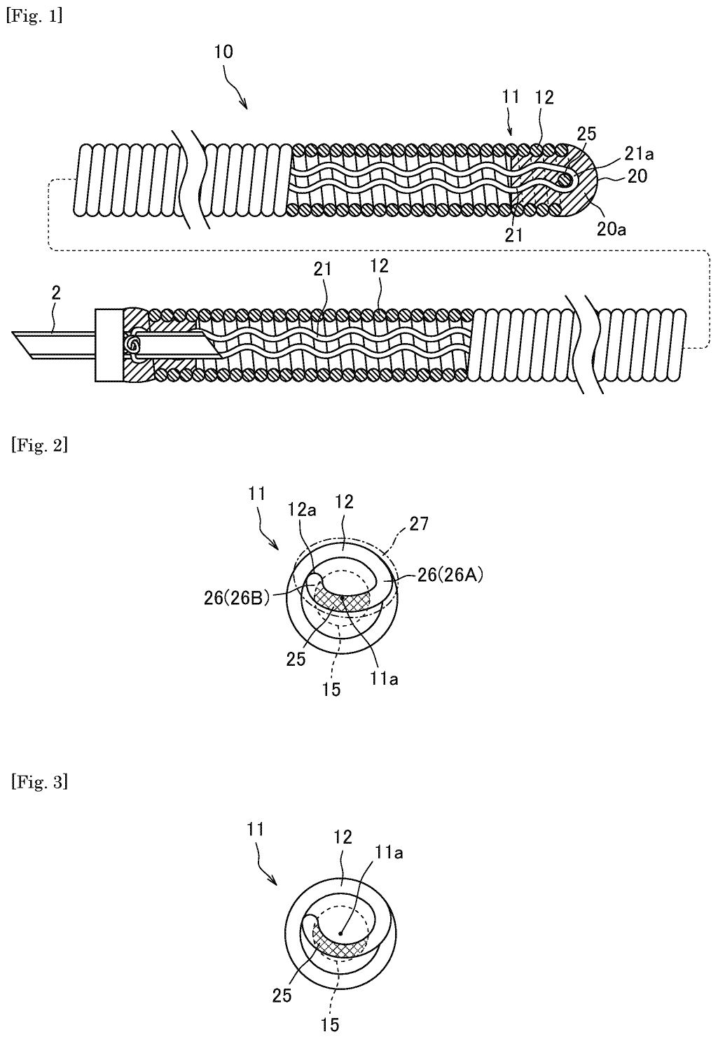

As long as the connecting part 25 of the wire 12 exists within the central region 15, as illustrated in FIG. 3, the wire 12 may exist on an outer side of the center of the central region 15 at the connecting part 25 in the view of the coil 11 from the distal side. In this case as well, the stretch resistant member 21 is readily disposed in the central region 15.

As illustrated in FIG. 2, the tip end 12a of the wire 12 preferably exists outside the central region 15. Since a part proximal to the tip end 12a of the wire 12 is disposed in the central region 15, it is difficult for the stretch resistant member 21 that is connected to the connecting part 25 of the wire 12 to fall off from the wire 12.

As illustrated in FIG. 2, in the view of the coil 11 from the distal side, the wire 12 includes a part that has a form of a closed curve shape, and an area surrounded by the closed curve is preferably 75% or less of an area surrounded by the circumference of the coil 11, more preferably 70% or less, and yet more preferably 60% or less, and is 30% or more or 40% or more of the area surrounded by the circumference of the coil 11 as acceptable area. With such a configuration, the stretch resistant member 21 is connected to the part of the wire 12 that is formed in a closed curve shape (closed curve line part 27), whereby it becomes difficult for the stretch resistant member 21 to fall off from the connecting part 25. The closed curve refers to a curved line in which one end part or a part of the curved line overlaps another part of the curved line. A maximum diameter part of the region surrounded by the curved line preferably substantially matches the amplitude of the wave of the stretch resistant member 21. In a case in which the maximum diameter part substantially matches the amplitude, the maximum diameter part of the region surrounded by the curved line is preferably larger or smaller by 25% than the amplitude of the wave of the stretch resistant member 21. The size of the maximum diameter part differs depending on the inner diameter of the coil 11; however, the size may be, for example, 25 μm or more, 30 μm or more, or 40 μm or more, or may be 100 μm or less, 80 μm or less, or 60 μm or less.

As illustrated in FIG. 2, a part of the closed curve line part 27 is preferably disposed in the central region 15. With such a configuration, the stretch resistant member 21 is also readily disposed in the central region 15. This allows the function of inhibiting the stretch resistant member 21 from extending in the axial direction of the coil 11 to be effectively exerted. The tip end 12a of the wire 12 may exist outside the central region 15.

The connecting part 25 is preferably disposed on the distal side of the coil 11. Specifically, the proximal end of the connecting part 25 is preferably disposed more distally than the position having a length of one tenth of the entire length of the coil 11 from the distal end of the coil 11 toward the proximal side, more preferably disposed more distally than the position having a length of one fifteenth, and yet more preferably disposed more distally than the position having a length of one twentieth. By setting the position of the connecting part 25 in this way, the range of the stretch resistant member 21 existing in the coil 11 can be made longer, and the flexibility of the in-vivo indwelling instrument 10 can be secured.

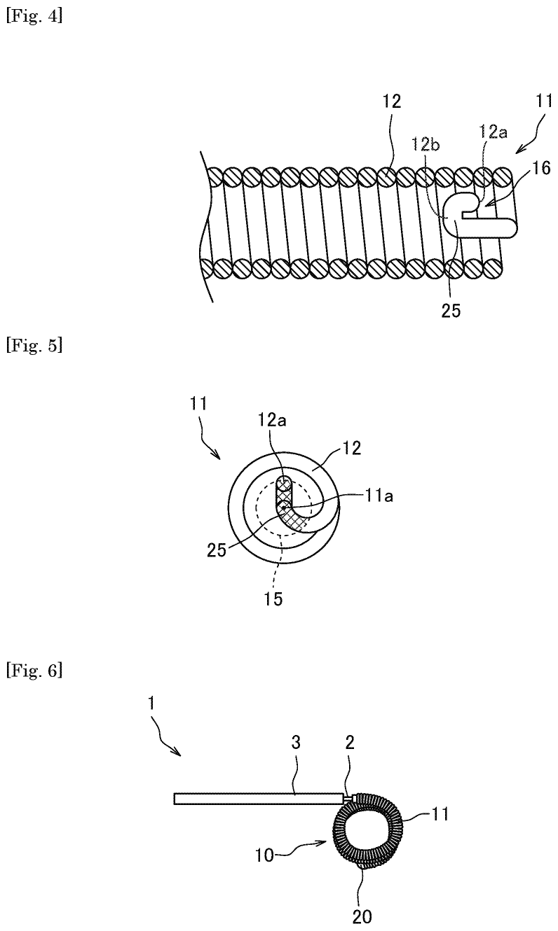

Next, a modification example of a coil 11 will be described with reference to FIGS. 4 and 5. FIG. 4 is a side view of the coil 11 according to an embodiment of the present invention. FIG. 5 is a front view of the coil 11 illustrated in FIG. 4. It is preferable that the connecting part 25 is a part in which a wire 12 is folded back in a shape of a hook along a distal and proximal direction of the coil 11. Specifically, it is preferable that a tip end 12a of the wire 12 is directed toward the distal side, and a folded part 12b of the wire 12 is directed toward the proximal side. Forming the hook 16 in this way also allows a stretch resistant member 21 to be readily connected to the connecting part 25. As a result, the stretch resistant member 21 is also readily disposed in the central region 15. This allows the function of inhibiting the stretch resistant member 21 from extending in the axial direction of the coil 11 to be effectively exerted. Although not illustrated, the wire 12 may be folded back along a radial direction of the coil 11 at the connecting part 25 and thereby a hook may be formed.

The tip end 12a of the wire 12 may exist outside the central region 15 as illustrated in FIGS. 2 and 3, or may exist inside the central region 15 as illustrated in FIG. 5. In the mode in which the large curvature part 26 or the closed curve line part 27 is provided in the wire 12, or in the mode in which the tip end 12a of the wire 12 is in contact with a wound part of the coil 11, the tip end 12a of the wire 12 exists outside the central region 15. On the contrary, in the mode in which the hook 16 is formed in the wire 12, the tip end 12a of the wire 12 exists within the central region 15. In any of the modes, the stretch resistant member 21 is readily disposed in the central region 15, and it is possible to inhibit the coil 11 from extending in the axial direction.

The stretch resistant member 21 and the connecting part 25 of the wire 12 are preferably bonded to the tip member 20, and is more preferably embedded within the tip member 20. This makes it possible to further inhibit the stretch resistant member 21 from falling off from the connecting part 25.

A configuration example of the in-vivo indwelling instrument delivering system 1 will be described with reference to FIG. 6. FIG. 6 is a side view of the in-vivo indwelling instrument delivering system 1 according to an embodiment of the present invention. As illustrated in FIG. 6, the in-vivo indwelling instrument delivering system 1 preferably includes an in-vivo indwelling instrument 10, a detachment part 2 connected to a proximal end part of the in-vivo indwelling instrument 10, and a pusher part 3 connected to the coil 11 of the in-vivo indwelling instrument 10 through the detachment part 2.

The detachment part 2 is not particularly limited as long as the in-vivo indwelling instrument 10 is detachable from the pusher part 3. For example, the detachment part 2 may be a wire shaped member or a rod-shaped member. The detachment part 2 may be made of a resin or a metal material. Examples of a resin constituting the detachment part 2 include hydrophilic resin of a synthetic polymer material such as polyvinyl alcohol (PVA), PVA cross-linked polymer, PVA water-absorbing gel freeing/defrosting elastomer, polyvinyl alcohol polymer such as ethylene-vinyl alcohol copolymer.

Examples of a detaching method of the in-vivo indwelling instrument 10 and the pusher part 3 include a method of causing the detachment part 2 to dissolve chemically, electrically, or thermally, a method of pushing out the in-vivo indwelling instrument 10 by water pressure, a method of releasing a mechanical engagement, and a method of electrolyzing the detachment part 2. As a simplest method, a method of causing a resin wire rod as the detachment part 2 to dissolve by adding high frequency current is preferably used. In such a case, in order to melt the detachment part 2 that connects the in-vivo indwelling instrument 10 with the pusher part 3, a high frequency current device is preferably connected to the pusher part 3. The high frequency current device generates Joule heat at the distal end part of the pusher part 3, thereby making it possible to melt the detachment part 2 that connects the in-vivo indwelling instrument 10 with the pusher part 3.

The pusher part 3 is a rod-shaped or wire-shaped member that retains the in-vivo indwelling instrument 10 and pushes the in-vivo indwelling instrument 10 toward the distal side. Examples of the pusher part 3 include a wire member, a coil member, or a combination thereof. The pusher part 3 can be made of, for example, a conductive material such as stainless steel. In order to grasp the position of the in-vivo indwelling instrument 10 in a body, an X-ray imaging marker may be provided in the pusher part 3. It is preferable that the X-ray imaging marker is provided to at least one of the distal end part or the proximal end part of the pusher part 3. The imaging marker may be in a ring shape or in a coil shape. A protection layer may be provided on an outer surface of the pusher part 3 in order to improve sliding between the pusher part 3 and another member such as a catheter. The protection layer is preferably made of a fluorine-based resin such as polytetrafluoroethylene (PTFE).

This application claims priority to Japanese Patent Application No. 2017-147759, filed on Jul. 31, 2017, and Japanese Patent Application No. 2017-147759, filed on Jul. 31, 2017, and the entire contents of which are incorporated by reference herein.

### REFERENCE SIGNS LIST

1: in-vivo indwelling instrument delivering system
2: detachment part
3: pusher part
10: in-vivo indwelling instrument
11: coil
11a: middle point of the maximum outer diameter of the coil
12: wire
12a: tip end of the wire
12b: folded part of the wire
15: central region
16: hook
20: tip
20a: tip end part of the tip
21: stretch resistant member
21a: folded part of the stretch resistant member
25: connecting part
26, 26A and 26B: large curvature part
27: closed curve line parts

1. An in-vivo indwelling instrument, comprising:
a coil that is formed by winding a wire to form a lumen of the coil, so that the coil extends from a proximal end to a distal end; and
a stretch resistant member that is placed in the lumen of the coil, wherein
the wire has a connecting part, to which the stretch resistant member is connected, at the distal end of the coil, the connecting part being located within a central region of the coil, and
the central region is a region defined by a circle having a diameter of half a maximum outer diameter of the coil and having a center that is a middle point of the maximum outer diameter in the view of a longitudinal direction of the coil.

2. The in-vivo indwelling instrument according to claim 1, wherein the connecting part is disposed at a position within a distance of one-tenth of an entire length of the coil from the distal end of the coil toward the proximal end.

3. The in-vivo indwelling instrument according to claim 1, wherein the wire includes a curved part having a curvature radius smaller than a curvature radius of the coil, and the connecting part is located between an end of the wire and the curved part at the distal end.

4. The in-vivo indwelling instrument according to claim 1, wherein, in the view of the coil from the distal end, the wire includes a part that has a form of a closed curve, and an area surrounded by the closed curve is 75% or less of an area surrounded by a circumference of the coil.

5. The in-vivo indwelling instrument according to claim 1, wherein the connecting part has a hook in which the wire is folded back in a shape of a hook along the longitudinal direction of the coil.

6. The in-vivo indwelling instrument according to claim 1, wherein a tip end of the wire at the distal end exists outside the central region of the coil.

7. The in-vivo indwelling instrument according to claim 1, wherein a tip end of the wire at the distal end exists inside the central region of the coil.

8. The in-vivo indwelling instrument according to claim 1, wherein, in the view of the coil from the distal end, a part of the wire forming the connecting part extends through the center of the central region.

9. The in-vivo indwelling instrument according to claim 1, wherein the stretch resistant member has a wave shape.

10. The in-vivo indwelling instrument according to claim 9, wherein an amplitude of the wave shape of the stretch resistant member is equal to or larger than an outer diameter of the wire.

11. The in-vivo indwelling instrument according to claim 1, further comprising a tip member, which is connected to the distal end of the coil.

12. The in-vivo indwelling instrument according to claim 11, wherein the tip member is made of an ultraviolet curing resin.

13. An in-vivo indwelling instrument delivering system, comprising:
the in-vivo indwelling instrument according to claim 1;
a detachment part connected to a proximal end part of the in-vivo indwelling instrument; and
a pusher part connected to the coil of the in-vivo indwelling instrument through the detachment part.
